(19) United States
(12) Patent Application Publication
(10) Pub. No.: US 2014/0154195 A1
(43) Pub. Date: Jun. 5, 2014

(54) Topical Compositions Containing White Strawberry Extract

(71) Applicants: Fatemeh Mohammadi, Hauppauge, NY (US); Nadine A. Pernodet, Hontington Station, NY (US); Donald F. Collins, Plainview, NY (US)

(72) Inventors: Fatemeh Mohammadi, Hauppauge, NY (US); Nadine A. Pernodet, Hontington Station, NY (US); Donald F. Collins, Plainview, NY (US)

(21) Appl. No.: 13/875,169

(22) Filed: May 1, 2013

Related U.S. Application Data
(60) Provisional application No. 61/641,015, filed on May 1, 2012.

(51) Int. Cl. A61K 8/97; A61Q 5/12; A61Q 1/10; A61Q 5/02; A61K 8/42; A61K 8/58

(52) U.S. Cl. CPC A61K 8/97; A61K 8/42; A61K 8/585; A61Q 1/10; A61K 8/975; A61Q 5/02; A61Q 5/12
USPC 424/63; 424/765; 424/195.15; 424/74

(57) ABSTRACT

Topical skin care composition comprising an extract of white strawberry and at least one additional skin care active.

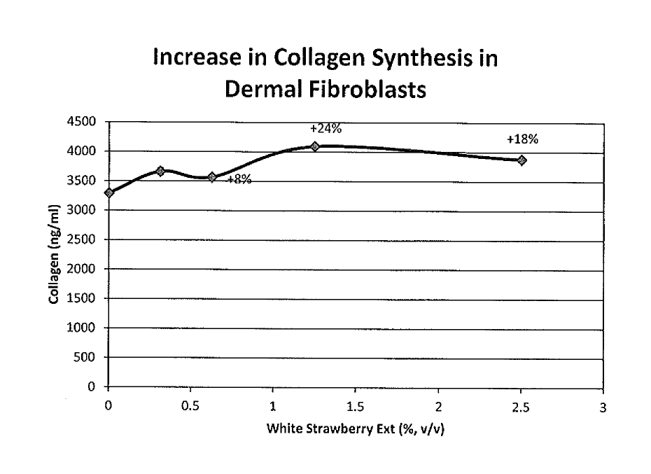

### CROSS-REFERENCE TO RELATED APPLICATIONS

This application claims priority from U.S. Provisional Patent Application Ser. No. 61/641,015, filed May 1, 2012.

### TECHNICAL FIELD

The invention is in the field of topical cosmetic and pharmaceutical compositions containing an extract from the genus Fragaria, more specifically a cultivar hybrid of various species in the genus including Fragaria chiloensis, Fragaria virginiana, or Fragaria Vesca also known as white strawberry or pineberry.

### BACKGROUND OF THE INVENTION

White strawberries are well known in horticulture. They are a hybrid cultivar of one or more different species of Fragaria genus. White strawberries look like strawberries except they are white instead of red and show small red flecks in the outer flesh. White strawberries, also referred to as pineberries, are said to have a taste somewhat like pineapple.

However, use of extracts from white strawberries in cosmetics, particularly skin care products, is not known. It has been discovered that the extract of white strawberry, when combined with an additional skin care actives, provide compositions that have improved efficacy in treating skin redness, irritation, allergies, as well as the conditions associated with aging skin such as wrinkles, fine lines, laxity, skin mottling, yellowing, or loss of collagen or elastin.

### SUMMARY OF THE INVENTION

The invention is directed to a topical skin care composition comprising an extract of white strawberry and at least one additional skin care active ingredient.

The invention is further directed to a method for treating skin to ameliorate the adverse effects of skin redness, irritation, allergies, wrinkles, fine lines, laxity, skin mottling, yellowing, or loss of collagen or elastin by applying a composition comprising an extract of white strawberry in combination with at least one additional skin care active.

### DESCRIPTION OF THE DRAWING

The FIGURE depicts the increase in collagen synthesis in dermal fibroblasts after treatment with the extract composition of the invention.

### DETAILED DESCRIPTION

The topical skin care composition of the invention may be present in the aqueous gel or solution, emulsion, or anhydrous form and may be in the form of creams, lotions, gels, sticks, cakes, powders, and the like. The term “skin” means both facial and body skin as well as lips.

If the compositions of the invention are present in an aqueous gel, solution, or suspension form, preferably such compositions will comprise from about 0.1 to 99%, preferably from about 0.5 to 90%, preferably from about 1 to 85% by weight of the total composition of water in combination with one or more of the other ingredients mentioned herein. If the compositions of the invention are in the form of emulsions, such emulsions may comprise from about 0.01 to 99%, preferably from about 0.05 to 95%, preferably from about 0.1 to 85% by weight of the total composition of water and from about 0.01 to 99%, preferably from about 0.05 to 95%, preferably from about 0.1 to 85% by weight of the total composition of oil, Such emulsions may be in the form of water in oil or oil in water emulsions. If the compositions are in the anhydrous form, they may comprise from about 0.01 to 99% oil and optionally from about 0.01 to 95% of one or more structuring agents or from about 0.01 to 95% of one or more particulates. All percentages mentioned herein are percentages by weight unless otherwise indicated.

### I. The White Strawberry Extract

The topical skin care composition of the invention comprising at least one extract of white strawberry. The extract may be present in amounts ranging from about 0.0001 to 55%, preferably from about 0.001 to 45%, more preferably from about 0.005 to 40% by weight of the total composition. The extract may be prepared by water, alcohol, or aqueous alcoholic extraction of the fruit, peel or seeds of the white strawberry. Most preferably, the extract is prepared from the fruit of the white strawberry. White strawberry extract may also be purchased from Toyatsu Chemiplas Corporation as an extract comprising about 18% white strawberry (Fragaria chiloensis) extract, about 30% butylene glycol, and about 52% water, all percentages by weight of the total extract. Preferred is where the white strawberry extract composition has a pH of 3.5 to 5.5. In vitro tests performed by the vendor purportedly show that extracts from white strawberries have anti-oxidant activity and may also have activity in inhibiting tyrosinase and stimulation of collagen synthesis in keratinocytes.

### II. Skin Care Active

The composition of the invention may contain one or more additional skin care actives. If present, the skin care active may be from about 0.0001-40%, preferably from about 0.0005-35%, more preferably from about 0.001-25%. Suitable skin care actives include, but are not limited to, anti-acne agents, anti-oxidants, astringents, exfoliants, analgesics, humectants, skin bleaching agents, skin conditioning agents having emollient, humectant, occlusive, or other conditioning functions, skin protectants, sunscreens and the like. Such skin care actives include, but are not limited to those set forth herein.

A. Vitamins or Vitamin Derivatives

Skin care actives include vitamins such as A, E, D, C, K, or derivatives thereof. For example, Vitamin C or ascorbic acid may be suitable, as well as fatty acid esters of ascorbic acid, amine, sulfonate, or phosphate derivatives thereof. Suitable fatty acid esters of ascorbic acid include ascorbyl palmitate, ascorbyl lactate, ascorbyl linoleate, ascorbyl stearate, ascorbyl tetraisopalmitate, ascorbyl dipalmitate. Suitable amino derivatives of ascorbic acid are as set forth in U.S. Pat. No. 5,916,915, which is hereby incorporated by reference in its entirety. On example of an amino phosphate derivative that is particular suitable is aminopropyl ascorbyl phosphate. Also suitable are sugar or polyol derivatives of ascorbic acid such as ascorbyl glucoside, and the like.

Also suitable are Vitamin E (tocopherol) and derivatives thereof, including but not limited to tocopheryl acetate, tocopheryl linoleate, tocopheryl oleate, tocopheryl acetate, tocopheryl nicotinate, tocopheryl retinoate, tocopheryl succinate, as well as alkoxylated derivatives thereof such as tocophereth 5, 10, 12, 18, or 50, with the number indicating the number of repeating ethylene oxide units.

Suitable anti-oxidants also include retinoids such as retinol, retinol esters, or retinoic acid. Suitable retinol esters include retinyl palmitate, retinyl stearate, and so on.

B. Anti-Acne Agents

Suitable anti-acne agents include benzoyl peroxide, salicylic acid, resorcinol or derivatives thereof. Also suitable are certain botanical extracts or ferments that contain components that have anti-acne activity, including but not limited to Actinidia arguta fruit extract, Candida bombicola/glucose/methylrapeseedate ferment, Citronellic acid, Curcuma xanthorrhiza root extract, Ginkgo leaf terpenoids, peat, and so on.

C. Anti-Oxidants

Suitable anti-oxidants are often found in the form of botanical extracts, including but not limited to Acacia Catechu wood extract, Acacia Victoriae extract, Acanthopanax Senticosis root extract, Acer Palmatum leaf extract, Achillea Millefolium Flower Extract, Aesculus Hippocastanum (Horse Chestnut) Extract, Agastache Rugosa Extract, Agrimonia Eupatoria Root Extract, Ajuga Reptans Cell Culture Extract, Ajuga Reptans Cell Culture Extract, Alchemilla Vulgaris Leaf Extract, Allium Cepa (Onion) Root Extract, Allium Fistulosum Root Extract, Allium Odorum, Seed Extract, Alpinia Uraiensis Stalk/Leaf Water, Angelica Furcijuga Flower/Leaf/Stem Extract, Angelica Keiskei Extract, Apium Graveolens (Celery) Seed Extract, Arabidopsis Thaliana Extract, Arbutus Unedo Fruit Extract, Arctium Lappa Fruit Extract, Artemisia Maritima Extract, Asarum Heterotropoides Extract, Asarum Heterotropoides Rhizome Extract, Avena Sativa extract, rice bran extract, Bambusa Vulgaris shoot extract, Beta Vulgaris extract, Betula Ermanii extract, Borago Officinalis extract, Botrytis Ferment extract, Calophyllum Inophyllum seed oil, Capparis Moonii Fruit extract, Capsella Brusa Pastoris Sprout water, Capsicum Annum fruit extract, Carpinus Laxiflora stem extract, Parinus Tschonoskii stem extract, Castanopsis Cuspidata stem extract, Cayaponia Tayuya, Selaginella tamariscina, Betula alba extract, Celosia, Cristata extract, Cercis Chinensis extract, Cereus Granidflora extract, Chaenomeles Sinensis fruit extract, Garcinia Mangostana extract (mangostin), Helianthus Annus extract, Hordeum Vulgare, Cucumis Sativus extract, Phyllanthus Emblica extract, Inonotus Obliquus extract, extensin, glycyrrhetinic acid, and those set forth on pages 2755-2757 of the C.T.F.A. International Cosmetic Ingredient Dictionary and Handbook, Eleventh Edition, 2006, which is hereby incorporated by reference in its entirety. In one preferred embodiment the anti-oxidant is Garcinia Mangostana extract, also referred to as Mangostin, Curcumin, Camellia sinensis, and the like.

D. Astringents

Suitable skin care actives include astringents such as ethanol, isopropyl alcohol, and botanical extracts from the genuses Acaica, Agrimonia, Anastatica, Angelica, Avena, Betula, Brassica, Citrus, Gingko, Lycium, Mentha, Morinda, Passiflora, Prunus, Rosa, Rubus, Vaccinium, Vitis, and the like.

E. Exfoliants

Suitable skin care actives include one or more exfoliants. Examples include alpha hydroxy acids such as glycolic, citric, malic, tartaric, lactic, maltobionic, lactobionic acids, including sodium salts thereof, and the like. Also suitable are honey, and derivatives thereof.

F. Skin Bleaching Agents

Also suitable as skin care actives are various types of skin bleaching agents, that is, agents that will whiten or bleach skin. Examples include ascorbyl glucoside, hydroquinone, Lactobacillus extract, Triticum vulgare germ extract, Prunus mume bud extract, and the like.

G. Skin Soothing Agents

Also suitable as skin care actives are skin soothing agents, that is actives that reduce skin redness, irritation, or inflammation. Examples include botanical extracts from Garcinia genus, such as Garcinia mangostana, colloidal oatmeal, and the like.

H. Skin Conditioning Agents

A variety of skin conditioning agents may be suitable including those that have emollient, humectant, occlusive, or other functional properties in conditioning skin. Such agents may be in the form of liquids, semi-solids, or solids at room temperature, The skin conditioning agents may be naturally derived or synthetic and include but are not limited to those set forth herein.

(1). Silicones

Suitable silicones include nonvolatile silicones, both water soluble or water insoluble. Such silicones preferably have a viscosity ranging from about 5 to 1,000,000 centistokes (cst), preferably 10 to 800,000 cst at 25° C. Suitable water insoluble silicones include amine functional silicones such as amodimethicone; phenyl substituted silicones such as bisphenylhexamethicone, trimethylsiloxyphenyl dimethicone, phenyl trimethicone, or polyphenylmethylsiloxane; dimethicone, dimethicone substituted with C₂₋₃₀ alkyl groups such cetyl dimethicone, stearyl dimethicone, behenyl dimethcone, and the like.

Suitable nonvolatile silicones may have the following general formula:

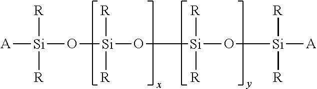

wherein R and R′ are each independently C₁₋₃₀ straight or branched chain, saturated or unsaturated alkyl, phenyl or aryl, trialkylsiloxy, and x and y are each independently 0-1,000,000; with the proviso that there is at least one of either x or y, and A is alkyl siloxy endcap unit, Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, stearyl dimethicone, behenyl dimethicone and the like wherein at least one R is a fatty alkyl (C₁₂, C₁₄, C₁₆, C₁₈, C₂₀, or C₂₂), and the other R is methyl, and A is a trimethylsiloxy endcap unit. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814. Stearyl dimethicone and behenyl dimethicone can also be purchased from Dow Corning Corporation or DeGussa Care & Surface Specialties under the trade name Abil.

(2). Esters

Also suitable as skin conditioning agent are mono-, di-, and triesters. The composition may comprise one or more esters selected from the group, or mixtures thereof.

(a). Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include, but are not limited to, hexyl laurate, butyl isostearate, hexadecyl isostearate, cetyl palmitate, isostearyl neopentanoate, stearyl heptanoate, isostearyl isononanoate, stearyl lactate, stearyl octanoate, stearyl stearate, isononyl isononanoate, and so on.

(b). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(c). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A Cosmetic Ingredient Dictionary and Handbook, Eleventh Edition, 2006, pages 2679 to 2688 which are hereby incorporated by reference in the entirety.

(3). Hydrocarbons

It may be desirable to incorporate one or more hydrocarbon oils into the composition as the skin conditioning agent. Suitable hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof.

(4). Glyceryl Esters of Fatty Acids

Also suitable as skin conditioning agents are synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, polyglyceryl-6 dioleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

### III. Other Ingredients

The composition of the invention may contain one or more additional ingredients including but not limited to those set forth herein.

A. Humectants

The composition of the invention may also contain one or more humectants. If present, suggested ranges are from about 0.001 to 50%, preferably from about 0.01 to 45%, more preferably from about 0.05 to 40% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, trehalose, and so on. Also suitable is urea or sugar derivatives, e.g. ethylhexylglycerin. In one preferred embodiment, the humectants used in the composition of the invention are C₁₋₆, preferably C₂₋₄ alkylene glycols, most particularly butylene glycol, and/or glycerin.

B, Surfactants

If desired, the compositions of the invention may contain one or more surfactants. This is particularly desirable when the composition is in the form of an aqueous gel, emulsion, either water in oil or oil in water. If present, the surfactant may range from about 0.001 to 50%, preferably from about 0.005 to 40%, more preferably from about 0.01 to 35% by weight of the total composition. Suitable surfactants may be silicone or organic, nonionic, anionic, amphoteric or zwitterionic. When the compositions of the invention are skin care compositions, it is preferred that the surfactants present be nonionic. Such surfactants include, but are not limited to, those set forth herein.

1. Silicone Surfactants

Suitable silicone surfactants include polyorganosiloxane polymers that have amphiphilic properties, for example contain hydrophilic radicals and lipophilic radicals. These silicone surfactants may be liquids or solids at room temperature.

(a). Dimethicone Copolyols or Alkyl Dimethicone Copolyols

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

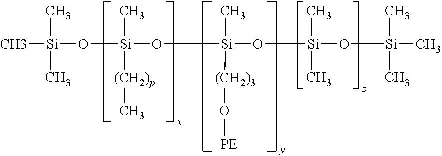

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 dimethicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

(b). Crosslinked Silicone Surfactants

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

2. Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable are ethoxylated propoxylated derivatives of C6-30 saturated or unsaturated fatty acids, for example, Di-PPG-2 myreth-10 adipate, Di-PPG-2 Ceteth-4 adipate, Di-PPG Myristyl Ether Adipate,

Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

C. Structuring Agents

In some cases it may be desired to incorporate one or more structuring agents into the composition. The term “structuring agent” means one or more ingredients that are capable of providing structure or viscosity to the compositions, If present, the structuring agents range from about 0.01 to 50%, preferably from about 0.5 to 45%, more preferably from about 0.5 to 35% by weight of the total composition. Structuring agents may structure or increase the viscosity of the aqueous phase or the oil phase, in the case where the composition comprises an oil phase. If a structuring agent is an oil phase structuring agent it means an ingredient or combination of ingredients that are soluble or dispersible in the oil phase, which will increase the viscosity, or structure, the oil phase. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suitable oil phase structuring agents include those that are silicone based or organic based. They may be polymers or non-polymers, synthetic, natural, or a combination of both.

1. Oil Phase Structuring Agents

(a). Silicone Structuring Agents

A variety of oil phase structuring agents may be silicone based, such as silicone elastomers, silicone gums, silicone waxes, linear silicones having a degree of polymerization that provides the silicone with a degree of viscosity such that when incorporated into the cosmetic composition it is capable of increasing the viscosity of the oil phase. Examples of silicone structuring agents include, but are not limited to:

(i). Silicone Elastomers

Silicone elastomers that may be used in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein, To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silsesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pala Kasei Kogyo KK, each of which are herein incorporated by reference in its entirety.

(ii). Silicone Gums

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million est. All ranges mentioned herein include all subranges, e.g. 550,000; 925,000; 3.5 million.

The silicone gums that are used in the compositions include, but are not limited to, those of the general formula wherein:

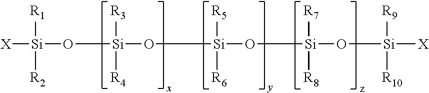

R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

(iii). Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

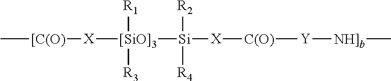

where X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

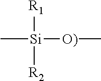

### and Y is:

(a) a linear or branched alkylene having from about 1-40 carbon atoms which may be substituted with (i) one or more amide groups having the general formula R₁CONR₁, or (ii) C₅₋₆ cyclic ring, or (iii) phenylene which may be substituted with one or more C₁₋₁₀ alkyl groups, or (iv) hydroxy, or (v) C₃₋₈ cycloalkane, or (vi) C₁₋₂₀ alkyl which may be substituted with one or more hydroxy groups, or (vii) C₁₋₁₀ alkyl amines; or

(b) TR₅R₆R₇

wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

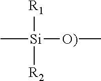

Preferred is where R₁, R₂, R₃, and R₁ are C₁₋₁₀, preferably methyl; and X and Y are linear or branched alkylenes. Preferred are silicone polyamides having the general formula:

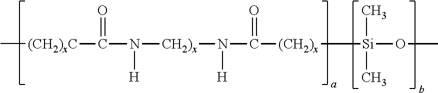

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

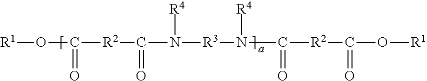

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C30-42 hydrocarbon; each R₃ is independently an organic group containing at least 2 carbon atoms, hydrogen atoms and optionally one or more oxygen or nitrogen atoms; and each R₁ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

(b). Organic Oil Phase Structuring Agents

(i). Natural or Synthetic Organic Waxes

Also suitable as the oil phase structuring agent may be one or more natural or synthetic waxes such as animal, vegetable, or mineral waxes. Preferably such waxes will have a higher melting point such as from about 60 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, oleic acid, stearic acid, and so on.

(ii). Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

(iii). Silicas and Silicates

Another type of structuring agent that may be used in the oil phase of the composition is silica, silicates, or silica silylate, and alkali metal or alkaline earth metal derivatives thereof. These silicas and silicates are generally found in the particulate form and include silica, silica silylate, magnesium aluminum silicate, and the like.

2. Aqueous Phase Structuring Agents

The structuring agent may be an aqueous phase structuring agent, that is an ingredient or combination of ingredients that will thicken the aqueous phase of the composition. Suitable aqueous phase structuring agents include Examples of such agents include various acrylate based thickening agents, natural or synthetic gums, and the like.

(a). Acrylate Polymers

For example, acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0, 1-99 parts of the A monomer, and about 0.1-99 parts of the B monomer, Acrylic polymer solutions include those sold by Seppic, Inc., under the tradename Capigel.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames.

Particularly suitable as the aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

(b). High Molecular Weight PEG or Polyglycerins

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

(c). Polysaccharides

Also suitable as aqueous phase thickening agents are various types of polysaccharides, such as xanthan gum, cellulose, dextrin, cyclodextrin, hydroxyethylcellulose, acacia gum, and the like.

D. Particulate Materials

The compositions of the invention may contain particulate materials in the form of pigments, inert particulates, or mixtures thereof. If present, suggested ranges are from about 0.1-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

1. Powders

The particulate matter may be colored or non-colored (for example white) non-pigmentatious powders. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

2. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

E. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

F. Film Forming Polymers

In certain cases it is desirable to include film forming polymers in the compositions of the invention. This is particularly the case if the compositions are in the form of colored cosmetic compositions, or where it is desired to ensure that the compositions adhere well to skin in.

If present, the polymers may range from about 0.01 to 85%, preferably from about 0.1 to 75%, preferably from about 0.5 to 40% by weight of the total compositions. Such polymers may silicones, copolymers of silicones and organic groups, or polymers or copolymers containing entirely organic groups. Examples of organic monomers that may be used to construct suitable film forming polymers include styrene, vinyl pyrrolidone, acrylic or methacrylic acid or its C₁₋₁₀ simple esters such as methyl methacrylate, methylacrylate, ethyl methacrylate, ethylacrylate, butyl acrylate, butyl methacrylate, and so on. The organic monomers may be neutralized with sales such as ammonia, sodium, potassium, and the like (e.g. ammonium acrylate, ammonium methacrylate, sodium acrylate, sodium methacrylate, and the like). The organic monomers may be copolymerized with other organic ingredients such as glycols, fatty acids, esters like those mentioned herein. Suitable film forming polymers also include copolymers of organic monomers and silicone, including a class of polymers generally referred to as silicone acrylate copolymers. One example of such polymers includes polydimethylsiloxane-g-polyacrylates sold by 3M Company under the tradename VS-70. Also suitable are silicone film forming polymers, including a group of silicones referred as silicone resins (trimethylsiloxysilicate or polymethylsilsesquioxane). A variety of film forming polymers may be used in the compositions of the invention depending on the desired end benefit.

G. Sunscreens

It may also be desirable to include one or more sunscreens in the compositions of the invention. Such sunscreens include chemical UVA or UVB sunscreens or physical sunscreens in the particulate form. Inclusion of sunscreens in the compositions containing the nicotinamide riboside will provide additional protection to skin during daylight hours and promote the effectiveness of the nicotinamide riboside on the skin.

1. UVA Chemical Sunscreens

If desired, the composition may comprise one or more UVA sunscreens. The term “UVA sunscreen” means a chemical compound that blocks UV radiation in the wavelength range of about 320 to 400 nm, Preferred UVA sunscreens are dibenzoylmethane compounds having the general formula:

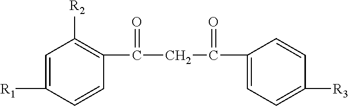

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Rouge under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

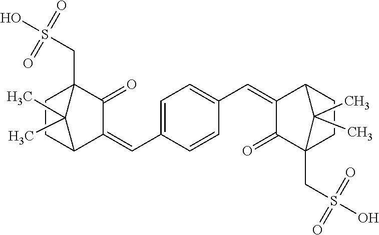 [SMILES: CC1(C)C2CCC1(CS(=O)(=O)O)C(=O)/C2=C\c1ccc(/C=C2\C(=O)C3(CS(=O)(=O)O)CCC2C3(C)C)cc1]

The composition may contain from about 0.001-20%, preferably 0.005-5%, more preferably about 0.005-3% by weight of the composition of UVA sunscreen, In the preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octociylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

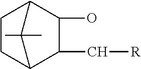

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

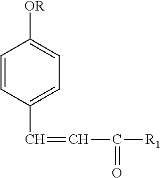

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the the general formula:

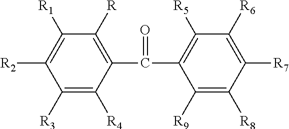

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

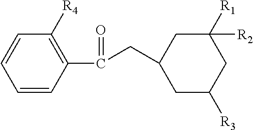

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

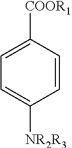

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have a certain SPF (sun protective factor) values ranging from about 1-100, preferably about from about 10 to 75 with ratios of UVA and UVB ranging from 1-3:1.

### IV. Forms of the Composition

The composition may be in the form of a cream, lotion, serum, gel, solution, stick, cake, powder, and so on. The composition may be a cleanser, toner, blush, skin cream or lotion, foundation makeup, concealer, eyeshadow, eyeliner, mascara, blush, lipstick, lip gloss, and the like.

For example, skin creams or lotions may be in the oil in water or water in oil emulsion form and contain, in addition to the amounts of water and oil mentioned herein in the ranges set forth, any one or more of the ingredients mentioned herein and in the amounts set forth.

Typical cleansers may contain from about 0.01-99% water and from about 0.1 to 95% of one or more surfactants, in addition to the other ingredients mentioned herein in the ranges set forth.

Toners are typically aqueous or aqueous-alcoholic and will comprise from about 0.1-99% water or a mixture of water and humectants or solvents such as ethanol, isopropanol, glycols, and the like, in addition to any of the ingredients mentioned herein in the ranges set forth.

The compositions of the invention may be in the form of foundation makeup or concealer. Typically such types of compositions contain from about 0.1 to 95% water, from about 0.1 to 80% oil, and from about 0.01 to 65% pigments. Foundations or concealers may also be in the anhydrous form and if so, may comprise from about 0.1 to 95% oil and from about 0.1-90% particulates.

In the case where the compositions are in the form of lipstick or lip gloss, typically they will comprise from about 0.1 to 95% oil, from about 0.1 to 85% structure agent, and from about 0.1 to 85% particulates.

The composition of the invention may be in the form of a sunscreen containing UVA or UVB sunscreens or mixtures thereof. Such sunscreens may be in the form of water in oil or oil in water emulsions or anhydrous compositions.

The composition of the invention may also be in the form of mascara or eyeliner. Mascaras are either anhydrous or in the emulsion form. If the former, the mascara may be present in a water in oil or oil in water emulsion form containing from about 0.1-95% water and from about 0.5 to 80% oil, in addition to one or more film forming polymers in the range of from about 0.1 to 80% and particulates in an amount ranging from about 0.1 to 70%.

Further embodiments of compositions within the scope of the invention include, but are not limited to:

Skin creams or lotions comprising, in addition to the White strawberry extract, from about 0.001-95% water, from about 0.01-90% of a skin protective emollient, from about 0.01-25% nonionic organic surfactants, from about 0.001-10% of one or more anti-oxidants, from about 0.01-25% of one or more humectants. Preferred is wherein the skin protective emollients are esters or silicones, the surfactants alkoxylated alcohols or derivatives thereof, the anti-oxidants including extracts of Garcinia Mangostana; and the humectants including glycerin or ethylhexyl glycerin.

In addition to the extract of White strawberry, skin toners include from about 0.001-99% water, from about 0.001-95% humectants such as glycerin or alkylene glycols, and optionally from about 0.001-30% of one or more anti-oxidants.

Skin cleansers include, in addition to the extract of White strawberry, from about 0.001-99% water, from about 0.001-60% surfactant, and optionally from about 0.001-95% humectants and optionally from about 0.001-30% of one or more anti-oxidants.

The composition may also be in the form of hair care products such as shampoos, conditioners, styling product's and the like. Typical shampoo products contain from about 0.1-95% water, 0.5-50% cleansing surfactant, and optionally various conditioning agent. Hair conditioner compositions comprise from about 0.1-95% water, 0.5-50% cationic conditioning agents, and optionally other ingredients.

### V. Methods, Kits, and Regimens

The invention also comprises a method for treating skin to ameliorate the adverse effects of irritation, inflammation, or allergic conditions comprising applying to the skin a composition containing an extract of white strawberry in combination with at least one skin care active ingredient.

The composition is preferably in the form of a skin cream or lotion. The composition may be applied to the skin one or more times per day, such as morning and evening prior to sleep.

The invention is also directed to a kit for treating irritated, inflamed, or allergic skin conditions comprising at least one of a cleanser or toner, and at least one topical skin care composition comprising an extract of white strawberry and at least one skin care active ingredient.

The invention is also directed to a regimen for treating irritated, inflamed, or allergic skin conditions comprising cleansing or toning the skin followed by application of a topical skin care composition comprising an extract of white strawberry and at least one skin care active ingredient.

The invention will be further described in connection with the following examples which are set forth for the purposes of illustration only.

### Example 1

A skin treatment composition is prepared as follows:

| Ingredient | w/w % |
|---|---|
| Hydrolyzed extension/butylene glycol | 0.50 |
| Propylene glycol dicaprate/Helianthus Annus (Sunflower) | 1.00 |
| seed cake/Hordeum Vulgare (Barley) extract/Cucumis Sativus | |
| (Cucumber) extract | |
| Hydrogenated lecithin | 0.02 |
| Beeswax | 0.20 |
| Coco caprylate/caprate | 2.30 |
| Aminopropyl ascorbyl phosphate | 0.01 |
| Hydroxyapatite | 0.50 |
| Trehalose | 0.10 |
| Dimethicone | 1.00 |
| Glyceryl stearate/PEG-100 stearate | 2.30 |
| Di-PPG-2 Myreth-10 adipate | 2.50 |
| Phyllanthus Emblica fruit extract | 0.03 |
| Glycyrrhetinic acid | 0.09 |
| Phenoxyethanol | 0.33 |
| Disodium EDTA | 0.06 |
| Cetearyl alcohol | 7.75 |
| Ethylhexylglycerin | 030 |
| Inonotus Obliquus (Chaga Mushroom) extract/cellulose | 0.03 |
| Phenoxyethanol/chlorphenesin/glycerin/sorbic acid | 0.95 |
| Zeolite | 0.50 |
| Glyceryl stearate | 1.20 |
| Butylene glycol | 7.70 |
| Garcinia Mangostana peel extract | 0.02 |
| Olive oil | 0.11 |
| Xanthan gum | 0.20 |
| White strawberry extract | 0.10 |
| Ferulic acid | 0.01 |
| Caprylic/capric triglyceride | 4.00 |
| Paraffin | 2.80 |
| Ceramide-2 | 0.10 |
| Methyl glucose dioleate | 0.60 |
| Sodium hyaluronate | 0.01 |
| Simethicone | 0.00005 |
| Shea butter (Butyrospermum Parkii) | 1.00 |
| Methyl glucose sesquistearate | 0.60 |
| Polyglyceryl-6 dioleate | 2.30 |
| Petrolatum | 5.00 |
| Glycerin | 5.00 |
| Colloidal Oatmeal/glycerin | 0.50 |
| Sorbitol | 1.00 |
| 7-dehydrocholesterol | 0.05 |
| Water | QS 100 |

The composition is prepared by combining the ingredients and mixing well to form a skin cream.

### Example 2

A primer composition for application to the face, either alone or under makeup, was made as follows:

| Ingredient | % by weight |
|---|---|
| Water | QS 100 |
| Acrylates crosspolymer | 3.00 |
| Glycerin | 15.00 |
| Sodium polyacrylate starch | 0.15 |
| Mangifera indica (Mango) seed butter | 2.00 |
| Dimethicone/vinyl dimethicone crosspolymer | 10.00 |
| PEG-10 dimethicone | 0.50 |
| Caprylyl glycol | 0.10 |
| Laminaria digitata extract/glucose | 0.20 |
| Water/glucose oxidase/lactoperoxidase | 0.01 |
| Perfluorhexane | 10.00 |
| Titanium dioxide/galactoarabinan | 1.68 |
| Iron oxide yellow | 0.18 |
| Iron oxide red | 0.08 |
| Iron oxides black | 0.03 |
| White strawberry extract | 1.00 |

The composition was prepared by separately combining the oil and water phase ingredients, then mixing well to emulsify.

### Example 3

White strawberry extract from Toyatsu Chemiplas Corporation (About 18% white strawberry (Fragaria chiloensis) extract, about 30% butylene glycol, and about 52% water) was tested in a collagen assay to determine ability to stimulate collagen synthesis in dermal fibroblasts. Adult normal human dermal fibroblasts (NHDF) from Clonetics were seeded and grown to confluence in a 96 well plate (Corning/Costar flat bottom) prior to being treated. The extract was added at concentrations ranging from 0.3125, 0.625, 1.25 and 2.5% (v/v). The plate was incubated for 3 days at 37° C. 15% CO₂ before the supernatants were harvested and stored at −80° C. in siliconized tubes until the ELISA was performed. The PIP ELISA (Takara Mirus Bio, Code MK101) was performed as outlined in the protocols supplied by the manufacturer and the results were calculated from the standard curves. For the pro-collagen ELISA the supernatants were diluted 1:200. In this assay, TGFβ (@ 10 ng/ml) was run as a positive control. The results are set forth in the FIGURE. The chart illustrates that the White strawberry extract stimulates collagen synthesis in dermal fibroblasts at concentrations ranging from 0.5 to 3% v/v. The positive control, TGFβ (10 ng/ml), increased collagen synthesis by an average of 72%.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A topical skin care composition comprising an extract of white strawberry in combination with at least one additional skin care active.

2. The composition of claim 1 wherein the additional skin care active is a ceramide.

3. The composition of claim 1 wherein the skin care active is colloidal oatmeal.

4. The composition of claim 1 wherein the skin care active is an extract from Garcinia genus.

5. The composition of claim 1 wherein the skin care active is from the Inonotus genus.

6. The composition of claim 1 wherein the skin care active is dimethicone.

7. The composition of claim 6 wherein the dimethicone is present in the composition at equal to or greater than 1% by weight of the total composition.

8. The composition of claim 1 in the form of a shampoo, conditioner, skin cream, skin lotion, toner, cleanser, foundation makeup, primer, blush, mascara, eyeliner, eyeshadow, and the like.

9. The composition of claim 1 comprising white strawberry extract, at least two skin care actives selected from dimethicone, ceramide, acrylate crosspolymer, and colloidal oatmeal.

10. The composition of claim 9 wherein the skin care actives are a mixture of dimethicone and acrylate crosspolymer.

11. The composition of claim 9 wherein the skin care actives are a mixture of dimethicone and ceramide.

12. The composition of claim 9 wherein the skin care actives are a mixture of dimethicone, ceramide, and colloidal oatmeal.

13. A topical composition comprising at least one extract of white strawberry having a pH of 3.5 to 5.5, dimethicone/vinyl dimethicone crosspolymer and dimethicone copolyol.

14. The composition of claim 13 which is a primer composition further comprising pigments.

15. The composition of claim 14 further comprising glycerin in an amount greater than 5% by weight of the total composition.

16. A topical skin care composition comprising least one extract of white strawberry having a pH of 5.5 and present in the composition in amounts ranging from 0.0001 to and at least one skin care active which is ascorbic acid or fatty acid esters thereof.

17. The composition of claim 16 additionally comprising an extract from Garcinia genus.

18. The composition of claim 17 additionally comprising an extract from the Inonotus (Chaga) genus.

19. The composition of claim 18 wherein the extract from the Chaga genus is Chaga mushroom.

20. The composition of claim 19 additionally comprising dimethicone.
